(19) United States
(12) Patent Application Publication
(10) Pub. No.: US 2015/0209299 A1
(43) Pub. Date: Jul. 30, 2015

(54) DRUG-ELUTING MEDICAL DEVICES

(71) Applicant: The Johns Hopkins University, Baltimore, MD (US)

(72) Inventors: Zhiyong Xia, Rockville, MD (US); Chao-Wei Hwang, West Friendship, MD (US)

(21) Appl. No.: 14/607,106

(22) Filed: Jan. 28, 2015

Related U.S. Application Data
(60) Provisional application No. 61/932,926, filed on Jan. 29, 2014.

(51) Int. Cl. A61K 9/70; A61K 9/00; A61K 45/06; A61K 31/616; A61K 38/18; A61K 31/337; A61K 31/565; A61K 38/20; A61K 38/21

(52) U.S. Cl. CPC A61K 9/70; A61K 38/2006; A61K 9/0092; A61K 45/06; A61K 38/21; A61K 38/1841; A61K 38/1875; A61K 31/337; A61K 31/565; A61K 31/616

(57) ABSTRACT

A medical device includes a plurality of drug-eluting nanofibers directly or indirectly located over an outer surface of the medical device, or utilized independently as a tissue engineering scaffold. The plurality of drug-eluting nanofibers include one or more therapeutic agents. Additional embodiments include a fabric having a plurality of drug-eluting nanofibers, in which the plurality of drug-eluting nanofibers include one or more therapeutic agents

### CROSS-REFERENCE TO RELATED APPLICATIONS

This application claims priority to and the benefit of prior-filed co-pending U.S. Provisional Application No. 61/932,926, filed Jan. 29, 2014, the content of which is herein incorporated by reference in its entirety.

### TECHNICAL FIELD

The present disclosure relates generally to drug-eluting medical devices (e.g., tissue engineering scaffolds and stents) including a plurality of drug-eluting fibers thereon and methods for forming the same.

### BACKGROUND

Regenerative medicine has been receiving increased attention as an approach that can reverse tissue damage caused by ischemic or other insult. Tissue engineering scaffolds, including vascular scaffolds, have been used as constructs in which new tissue can be regenerated via in-growth of cells.

Most scaffolds are constructed of biodegradable polymer or materials derived from biological tissue (e.g., collagen). When tissue engineering scaffolds are applied directly in vivo, for example an engineered vascular scaffold applied in an artery, there are limitations that arise related to the ability of the scaffolds to attract the right types of cells while excluding other cell types, promote their rapid proliferation, and repopulation of the scaffold in an appropriate anatomic configuration. A scaffold which elutes appropriate chemoattractants and growth factors is therefore advantageous, as these biomolecules can induce certain desirable cell types to home in to the scaffold surface

In interventional cardiology, drug-eluting stents (DES) have been getting more attention, due to their anti-scaring capability as compared to bare metal stents (BMS). DES stents are generally fabricated via coating a BMS with a drug carrying media.

One class of DES stents utilize polymer encapsulated drug coating on the DES. The polymer coatings can be both non-biodegradable and biodegradable. These types of stents have improved restenosis compared to BMS; however, they also have exhibited a high rate of myocardial infarction and even mortality in some cases. Another class of DES stents are the so-called “structured stents”, which include micro-structured surfaces created on the surface of a BMS for drug incorporation. For example, textured 316 L stainless steel stents have been utilized. In other approaches, coupling agents have been used for anchoring a drug onto the stent surface. Such stents, however, have exhibited shedding of the coupling agent resulting in undesired particle debris. Moreover, neointimal hyperplasia has been found to increase with these types of stents.

As such, there at least remains a need in the art for improved drug-eluting medical devices (e.g., stents and tissue engineering scaffolds), which eliminate or at least mitigate the shortcomings associated the past devices.

### BRIEF SUMMARY

One or more embodiments of the invention may address one or more of the aforementioned problems. Certain embodiments according to the present invention provide devices comprising a medical device (e.g., a stent) and a plurality of drug-eluting nanofibers directly or indirectly located over an outer surface of the medical device. In certain embodiments, for example, the plurality of drug-eluting nanofibers comprise one or more therapeutic agents therein. In certain embodiments, the plurality of drug-eluting nanofibers comprise one or more biodegradable polymers. In this regard, certain embodiments of the present invention comprise a device which provides a controlled or sustained release of one or more therapeutic agents when positioned within a mammal.

In another aspect, embodiments of the present invention provides a fabric (e.g., a nonwoven fabric) comprising a plurality of drug-eluting nanofibers. In certain embodiments, the plurality of drug-eluting nanofibers comprise one or more therapeutic agents therein (e.g., within a core portion of sheath-core nanofibers, within an interior cavity of hollow nanofibers, within pores of porous nanofibers). In this regard, the fabric may comprise a drug-eluting fabric which may be formed into a variety of configurations (e.g., sleeve, tube, etc.) and attached and/or positioned onto or over the top of a medical device, or used independently as a tissue engineering scaffold. Drug-eluting fabrics according to certain embodiments may comprise a mixture of drug-eluting nanofibers and non-drug-eluting fibers. In certain embodiments, the nanofiber fabric may contain nanofibers that elute different drugs (e.g., one or more therapeutic agents) depending on the location within the fabric (e.g., different layers or different regions within a layer may elute differing therapeutic agent(s)). Since traditionally implanted scaffolds are typically limited in size and exposed surface area, scaffolds comprising a plurality of drug-eluting nanofibers, according to certain embodiments of the present invention, provide a significantly greater exposed surface area. In accordance with certain embodiments of the present invention, the nanofiber fabric may comprise (or be used) as a tissue engineering scaffold in which the drug elution profile of each individual fiber (or groups of fibers) can be separately controlled. The vastly increased drug-eluting surface area, according to certain embodiments of the present invention, increases the amount of attractant biomolecules that can be released. More importantly, because different fibers within the same structure can be made to release different biomolecules, such a device will allow different parts of the scaffolds to be populated with different celltypes, potentially accelerating the repopulation of the scaffold in an appropriate anatomic configuration.

In certain embodiments, the present invention comprises a drug-eluting fiber (e.g., a nanofiber). The drug-eluting fibers may be formed into a fabric as referenced above or provided as separate and discrete fibers.

In yet another aspect, example embodiments of the present invention provides methods for forming one or more drug-eluting fibers (e.g., nanofibers). In accordance with certain embodiments, the methods comprise forming a plurality of nanofibers and adding one or more therapeutic agents within the plurality of nanofibers. In certain embodiments, the addition of the one or more therapeutic agents may be added after formation of the nanofibers, while in some embodiments the addition of the one or more therapeutic agents may be added simultaneously with the formation of the nanofibers. In further embodiments, the present invention provides methods of forming drug-eluting fabrics formed from the plurality of drug-eluting nanofibers.

In another aspect, example embodiments of the present invention provides a method of making a medical device. Methods according to certain embodiments may comprise positioning a plurality of drug-eluting nanofibers directly or indirectly over an outer surface of a medical device (e.g., a stent). In certain embodiments, the positioning of the drug-eluting nanofibers may comprise directly depositing (e.g., spinning) the nanofibers onto the medical device or forming a network of drug-eluting nanofibers (e.g., a formed fabric) and covering a portion of the medical device with the network of drug-eluting nanofibers.

### BRIEF DESCRIPTION OF THE DRAWING(S)

Example embodiments of the present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

FIG. 1 illustrates drug-eluting nanofibers having a sheath-and-core configuration with therapeutic agents loaded in the core of the nanofibers according to certain embodiments of the present invention.

FIG. 2 illustrates a schematic of one process for forming nanofibers according to certain embodiments of the present invention.

FIG. 3 illustrates hollow nanofibers which can be loaded with one or more therapeutic agents according to certain embodiments of the present invention.

FIG. 4 illustrates drug-eluting nanofibers comprising a porous-outer surface comprising a plurality of pores containing therapeutic agent(s) loaded into the pores according to certain embodiments of the present invention.

FIG. 5 illustrates a cross sectional view of a medical device having a plurality of drug-eluting nanofibers positioned over an outer surface of the medical device according to certain embodiments of the present invention.

FIG. 6 illustrates the estimated diffusion kinetics of water diffusing through a 10 micron coating of a biodegradable material.

### DETAILED DESCRIPTION

Example embodiments of the present invention now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

Example embodiments of the present invention includes devices comprising a medical device and a plurality of drug-eluting nanofibers. Certain embodiments according to the present invention, for example, provide devices comprising a medical device (e.g., a stent) and a plurality of drug-eluting nanofibers directly or indirectly located over an outer surface of the medical device, or utilized independently as a tissue engineering scaffold. In certain embodiments, for example, the plurality of drug-eluting nanofibers comprise one or more therapeutic agents therein. In certain embodiments, the plurality of drug-eluting nanofibers comprises one or more biodegradable polymers. In this regard, certain embodiments of the present invention comprise a device which provides a controlled or sustained release of one or more therapeutic agents when positioned within a mammal.

The terms “polymer” or “polymeric”, as used herein, may comprise synthetic and/or biodegradable homopolymers, copolymers, such as, for example, block, graft, random, and alternating copolymers, terpolymers, etc., and blends and modifications thereof. They may also comprise the various topologies of polymers that are possible, including infinite networks, branched, star, brush, and linear. Furthermore, unless otherwise specifically limited, the term “polymer” shall include all possible geometrical configurations of the material. These configurations include, but are not limited to, isotactic, syndiotactic, and atactic symmetries.

The term “biodegradable” and “biodegradable polymer”, as used interchangeably herein, may comprise a polymer (as referenced above) which degrades as a result from the action of naturally occurring microorganisms such as bacteria, hydrolysis, algae or fungi. In certain embodiments, the biodegradable polymer may degrade by surface erosion of bulk degradation. They may also comprise polymers derived from lactic and glycolic acid, including but not limited to poly(dioxanone), poly(trimethylene carbonate) copolymers, and poly(ε-caprolactone) homopolymers and copolymers. In certain embodiments, the biodegradable polymer may comprise polyanhydrides, polyorthoesters, polyphosphazenes, or combinations thereof, which naturally breakdown or degrade within the body of a mammal. Additional exemplary biodegradable polymers, suitable for certain embodiments of the present invention, include hyaluronic acid, poly(lactic acid), poly(glycolic acid), poly(lactide-co-glycolide) copolymers, polyamide esters, polyvinyl esters, polyvinyl alcohol, and polyanhydrides.

As used herein, the term “layer” may comprise a region of a given material whose thickness is small compared to both its length and width. As used herein a layer need not be planar, for example, taking on the contours of an underlying substrate. A layer can be discontinuous (e.g., patterned).

The term “tissue”, as used herein, may comprise any component of human body, including, but not limited to, muscle, blood vessels, bone, fat tissue, or skin.

The term “therapeutic agent”, as used herein, may comprise biologically active materials, genetic materials, stem cells, and biological materials. Therapeutic agents, in certain embodiments of the present invention, may include their analogs and derivatives. Exemplary therapeutic agents may comprise one or more of a small molecular drugs, anti-thrombotic agents, anti-platelet agents, anti-angiogenic agents, anti-proliferative agents, proliferative agents, anti-restenosis agents, chemotactic agents, cell adhesion molecules, growth factors, paracrine factors, extracellular vesicles, exosomes, nucleic acids, genetic material, DNA, RNA, and/or micro-RNA.

Non-limiting examples of suitable therapeutic agents may include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazocin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, paclitaxel (as well as its derivatives, analogs or paclitaxel bound to proteins, e.g. Abraxane™) 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In certain embodiments, the therapeutic agent comprises a smooth muscle cell inhibitor or antibiotic. In yet another embodiment, the therapeutic agents comprises an antibiotic such as erythromycin, amphotericin, rapamycin, adriamycin, etc.

The term “genetic materials”, as used herein, may comprise DNA or RNA, including, without limitation, of DNA/RNA encoding a useful protein stated below, intended to be inserted into a mammalian body including viral vectors and non-viral vectors.

The term “biological materials”, as used herein, may comprise include proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), Skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIP-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. These dimeric proteins can be provided as homodimers, heterodimers, or combinations thereof, alone or together with other molecules. Other biological materials could include extracellular vesicles, such as exosomes.

Other non-genetic therapeutic agents, according to certain embodiments of the present invention, may include: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin; anti-inflammatory agents such as glucocorticoids, betamethasone, dexamethasone, prednisolone, corticosterone, budesonide, estrogen, sulfasalazine, rosiglitazone, mycophenolic acid and mesalamine; anti-neoplastic/anti-proliferative/anti-miotic agents such as paclitaxel, 5-fluorouracil cisplatin, vinblastine, vincristine, epothilones, methotrexate, azathioprine, adriamycin and mutamycin; endostatin, angiostatin and thymidine kinase inhibitors, cladribine, taxol and its analogs or derivatives; anesthetic agents such as lidocaine, bupivacaine, and ropivacaine; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides; DNA demethylating drugs such as 5-azacytidine, which is also categorized as a RNA or DNA metabolite that inhibit cell growth and induce apoptosis in certain cancer cells; vascular cell growth promoters such as growth factors, vascular endothelial growth factors (VEGF, all types including VEGF-2), growth factor receptors, transcriptional activators, and translational promoters; vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin, rapamycin (sirolimus); angiogenic substances, such as acidic and basic fibroblast growth factors, estrogen including estradiol (E2), estriol (E3) and 17-beta estradiol; drugs for heart failure, such as digoxin, beta-blockers, angiotensin-converting enzyme (ACE) inhibitors including captopril and enalopril, statins and related compounds; and macrolide agents such as sirolimus, pimecrolimus, tacrolimus, zotarolimus or everolimus.

In certain embodiments, the therapeutic agent may comprise anti-proliferative drugs, such as steroids, vitamins, and restenosis-inhibiting agents. Exemplary restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in certain embodiments of the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt. Other exemplary therapeutic agents, in certain embodiments, include tacrolimus; halofuginone; inhibitors of HSP90 heat shock proteins such as geldanamycin; microtubule stabilizing agents such as epothilone D; phosphodiesterase inhibitors such as cliostazole; Barket inhibitors; phospholamban inhibitors; and Serca 2 gene/proteins. In some embodiments of the present invention, the therapeutic agents may comprise nitroglycerin, nitrous oxides, nitric oxides, aspirins, digitalis, estrogen derivatives such as estradiol and glycosides.

The term “medical device”, as used herein, may comprise any medical device capable of being inserted into a mammalian (e.g., human) body and used in conjunction with a plurality of drug-eluting fibers (e.g., nanofibers). Medical devices may comprise stents, stent sleeves, pacemakers, tissue engineering scaffolds, vascular grafts, implantable cardioverter-defibrillators, pacemaker electrodes, implantable cardioverter-defibrillator leads, biventricular implantable cardioverter-defibrillator leads, artificial hearts, artificial valves, ventricular assist devices, balloon pumps, catheters, central venous lines, implants, or sensors.

### I. DRUG-ELUTING FIBERS AND FABRICS

In one aspect, the present invention provides drug-eluting fibers in which the fibers comprise one or more therapeutic agents. In certain embodiments of the present invention, the fibers comprise nanofibers including the one or more therapeutic agents. Nanofibers according to certain embodiments of the present invention may be comprised of one or more biodegradable polymers configured for degrading over a defined or desired period of time once placed inside a mammalian body.

In accordance with certain embodiments, the drug-eluting fibers comprise nanofibers comprising a sheath-and-core configuration. Such nanofibers, for instance, comprise a core and a sheath surrounding the core, in which the core comprises one or more therapeutic agents loaded therein. As noted above, the sheath-and-core drug-eluting fibers can comprise one or more biodegradable polymers. For example, the core and the sheath may each comprise a biodegradable polymer, in which the core may comprise the same or different biodegradable polymer as the sheath. In certain embodiments, the biodegradable polymer forming the sheath may naturally degrade at a faster rate than the biodegradable polymer forming the core and housing the therapeutic agent(s). In this regard, the nanofibers may be tailored by manipulation of the selection or particular blend of biodegradable polymers for the sheath and/or core of the nanofibers.

Sheath-and-core configured drug-eluting nanofibers may comprise an outer diameter of less than about 10,000 nm. In certain embodiments, the outer diameter may comprise from at least about any of the following: 1, 5, 25, 100, and 1000 nm and/or at most about 10000, 5000, 2500, and 1000 nm (e.g., about 1-10000 nm, about 1-1000 nm, etc.).

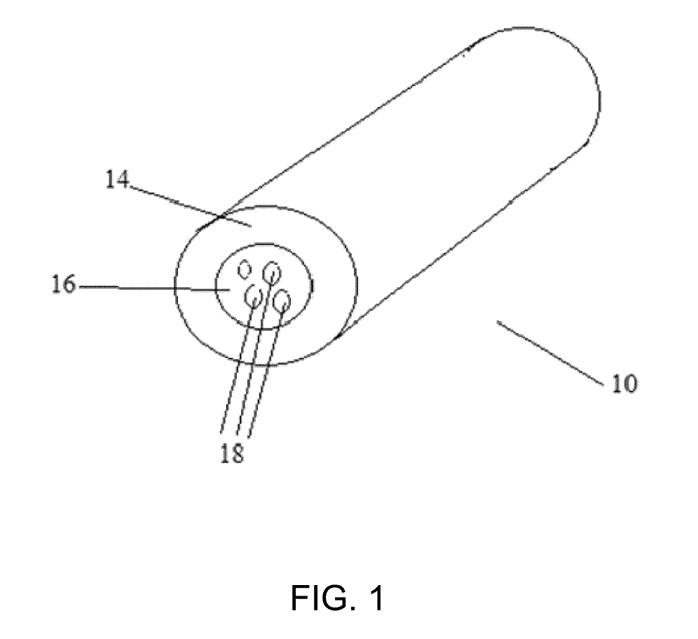

FIG. 1 illustrates sheath-and-core configured drug-eluting nanofibers, according to certain embodiments of the present invention. As illustrated by FIG. 1, the sheath-and-core configured drug-eluting nanofibers 10 comprise a sheath 14 comprising, for example, a first biodegradable polymer or blend of polymers and a core 16 comprising, for example, a second biodegradable polymer or blend of polymers. In certain embodiments of the present invention, the first biodegradable polymer or blend of polymers may be the same as or different than the second biodegradable polymer or blend of polymers. As also illustrated by FIG. 1, the sheath-and-core configured drug-eluting nanofibers may comprise one or more therapeutic agents 18 loaded within the core 16. In this regard, the second biodegradable polymer or blend of polymers may comprise or function as a carrier or matrix for housing the one or more therapeutic agents. In certain embodiments according to the present invention, for instance, the first biodegradable polymer or blend of polymers may be selected or configured to degrade at a faster rate than the second biodegradable polymer or blend of polymers when located within a mammalian body. In this regard, selection and/or appropriate configuration of the first and second biodegradable polymers or blends thereof can provide a tailored approach to realizing a sustained and/or controlled release of the one or more therapeutic agents. For instance, the degradation of the shell of the drug-eluting nanofibers may function as a time-delay before any therapeutic agent is released at all. For instance, it may be desirable for the delivery of the therapeutic agent(s) within the core to not begin until after a desired time frame (e.g., time associated with implanting the medical device in the appropriate location). In this regard, the first biodegradable polymer or blend of polymers may be tailored or configured to “protect” or “shield” the therapeutic-containing core for the desired time frame. Such embodiments, therefore, may provide a delayed-release of the one or more therapeutic agents.

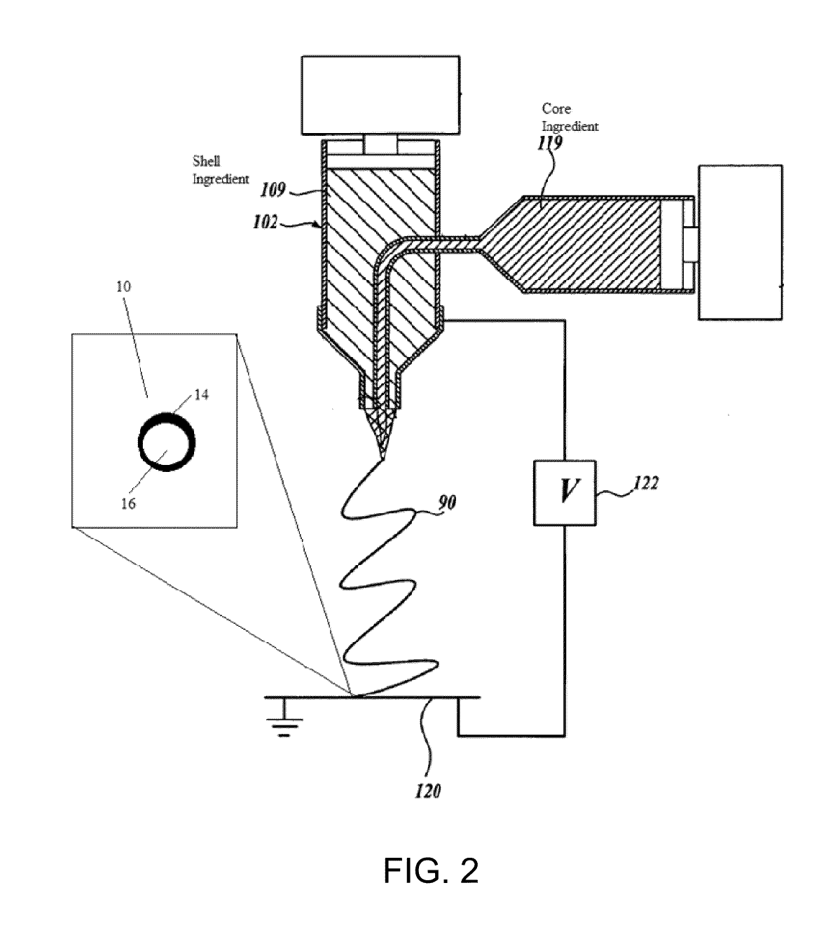

Although the method in which the sheath-and-core drug-eluting nanofibers is not particularly limited, a plurality of sheath-and-core drug-eluting nanofibers may be formed by an electrospinning process. For example, a co-axle needle or die (e.g., a spinneret) may be utilized to form a core-and-sheath configured nanofiber including one or more therapeutic agents loaded or housed within the biodegradable polymer defining the core of the nanofibers. As shown in FIG. 2, for example, the core-and-sheath configured drug-eluting nanofibers may be formed by utilizing a spinneret 102 (e.g., plastic syringe) having an outer capillary or needle defining a bore, the needle channeling a first liquid 109 (e.g., comprising a shell or core biodegradable polymeric material) in the spinneret and an inner capillary with a distal end inserted into the needle, in which the inner capillary channels a second liquid 119 (e.g., comprising a core biodegradable polymeric material) in the spinneret. A conductive collector 120, such as a piece of foil or a silicon wafer, may be provided a certain distance from the spinneret and a voltage 122 is applied between the needle and the collector. The first liquid may comprise a shell-biodegradable polymeric material that may include a biodegradable polymer, a solvent, and/or a sol-gel precursor. An acid stabilizer may also be included if desired. The second liquid may comprise a core-biodegradable polymeric material that may include a biodegradable polymer, a solvent, and/or a sol-gel precursor. An acid stabilizer may also be included if desired. The applied voltage is selected to be sufficiently high to induce electrospinning—that is, such that a jet of fluid 90 is ejected from the spinneret to the collector to form a composite nanofiber having a sheath-and-core configuration. As noted above, the sheath-and-core drug-eluting nanofibers, according to certain embodiments of the present invention, may be formed by any known method for producing nanofibers.

In accordance with certain embodiments, the core-biodegradable polymeric material may also include one or more therapeutic agents therein. For example, the one or more therapeutic agents may be added in situ to the second liquid referenced above. That is, for instance, the one or more therapeutic agents may be added to the second liquid (e.g., polymeric melt) that is channeled through the inner capillary channel to form the core of the drug-eluting nanofibers.

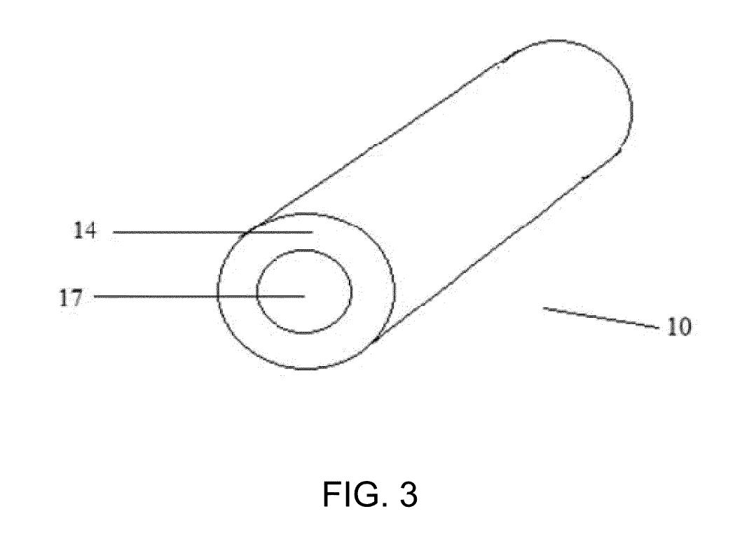

In certain embodiments, the drug-eluting nanofibers may comprise a hollow-fiber configuration comprising an outer wall defining an interior cavity. In accordance with certain embodiments, the hollow-fiber configured nanofibers can be formed in a variety of known methods. In some embodiments, for example, the hollow-fiber configured nanofibers may be formed utilizing an electrospinning process as illustrated in FIG. 2. In such embodiments, however, instead of utilizing a therapeutic-containing core, a soluble (e.g., water soluble) polymer may be used to form the core. In certain embodiments, the core may be formed from a water soluble polymer, such as polyethylene glycol (among others). Upon or after formation of the sheath-and-core nanofibers, the nanofibers may be washed with a suitable solvent to extract and/or dissolve the core component of the sheath-and-core nanofibers, while not extracting and/or dissolving the shell component. For example, the formed sheath-and-core nanofibers may be washed with de-ionized water, or tetrahydrofuran (THF) to extract, for example, a core comprising polyethylene glycol and leave a hollow structure inside the nanofiber. After dissolution and/or extraction of the core, the resulting nanofibers comprise a hollow-fiber configuration comprising an outer wall (e.g., the shell) defining an interior cavity. FIG. 3, for example shows nanofibers 10 comprising a hollow-fiber configuration including an outer wall or shell 14 and an interior cavity 17.

In accordance with certain embodiments, the interior cavity may comprise one or more therapeutic agents located therein. For instance, the interior cavity may comprise a therapeutic composition comprising the one or more therapeutic agents. In accordance with certain embodiments, the therapeutic composition comprises a solution, suspension, gel, solid-preparation or any combinations thereof. The interior cavity of the hollow-fiber configured nanofibers may be loaded with the therapeutic composition by, for example, soaking the hollow-configured nanofibers in a target therapeutic composition (e.g., a solution, suspension, etc.) followed by drying (or allowing to dry) the soaked hollow-configured nanofibers to provide drug-eluting nanofibers having a hollow-fiber configuration containing one or more therapeutic agents contained therein. In certain embodiments, the dried hollow-configured nanofibers may comprise a therapeutic composition comprising a solid, gel, or generally having a viscosity high enough that the therapeutic composition does not freely flow out of the hollow-configured nanofibers. In certain embodiments, however, the resulting drug-eluting nanofibers comprise one or more seals configured to temporarily contain the therapeutic composition within the interior cavity or cavities of the drug-eluting nanofibers. For example, the one or more seals may comprise a mechanical crimp, thermally formed seal, or combination thereof. In this regard, the seals may comprise discrete closures along the length of the drug-eluting nanofibers. As such the one or more seals (e.g., discrete closures) and the outer wall/shell 14 define substantially enclosed pocket portion(s) along the length of the drug-eluting nanofibers, in which the therapeutic agent may be contained. In this regard, the one or more seals may be formed after the interior cavity of the hollow-fiber configured nanofibers are loaded with the therapeutic composition.

Hollow-fiber configured drug-eluting nanofibers, according to certain embodiments of the present invention, may comprise an outer diameter of less than about 10,000 nm. In certain embodiments, the outer diameter may comprise from at least about any of the following: 1, 5, 25, 100, and 1000 nm and/or at most about 10000, 5000, 2500, and 1000 nm (e.g., about 1-10000 nm, about 1-1000 nm, etc.).

Drug-eluting nanofibers according to certain embodiments of the present invention may comprise a porous-outer surface comprising a plurality of pores located substantially about the outer surface of the nanofibers. The plurality of pores may, for example, comprise a general pit-like or bowl-like structure. In this regard, the plurality of pores may be particularly suitable for housing or containing a therapeutic composition (e.g., one or more therapeutic agents) therein. In accordance with certain embodiments, the plurality of pores comprise one or more therapeutic agents disposed therein.

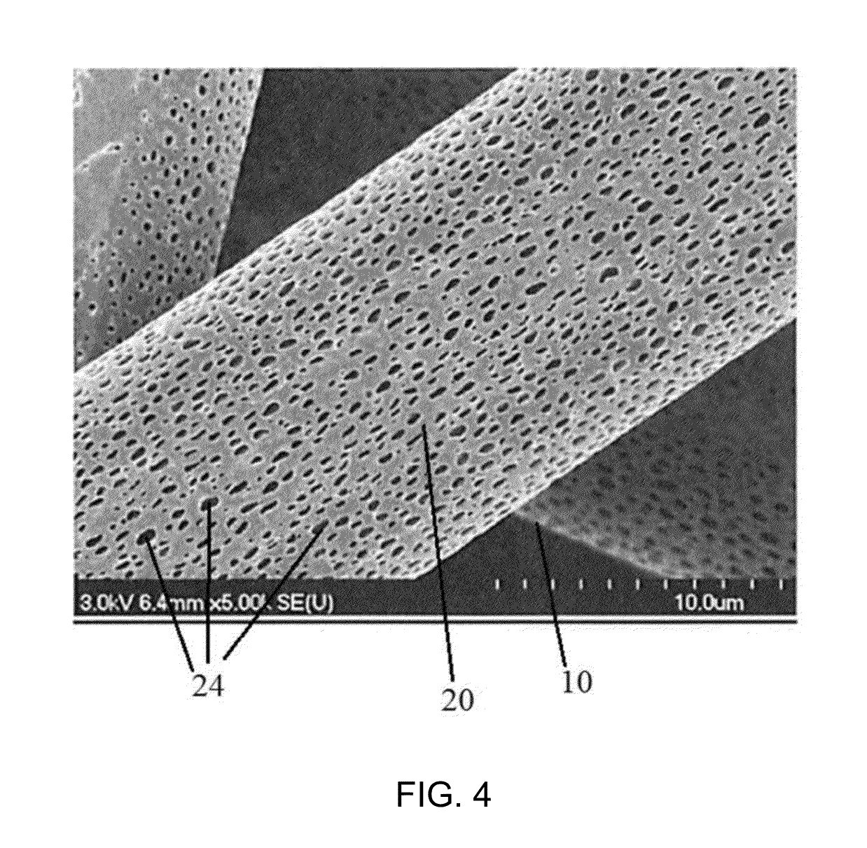

FIG. 4, for example, illustrates drug-eluting nanofibers 10 formed from poly(lactic-co-glycolic acid) (PLGA) and comprising a porous-outer surface 20 comprising a plurality of pores 24. Drug-eluting nanofibers according to certain embodiments of the present invention, such as those illustrated by FIG. 4, may be formed by a variety of methods, including an electrospinning process, melt fibrillation process, meltblown process, etc. By way of example only, drug-eluting nanofibers comprising a porous-outer surface comprising a plurality of pores may be formed by an electrospinning process in which a high vapor pressure organic solvent is included in the polymeric solution or melt that is spun into a plurality of nanofibers. In accordance with certain embodiments of the present invention, for instance, the high vapor pressure organic solvent comprises a vapor pressure, for example, of at least about 20 mm HG at 23° C. Exemplary, but not limiting, high vapor pressure organic solvents may comprise 1,2-dichloroethane (DCE), which has a vapor pressure of 60 mm Hg at 20° C., and methanol (MeOH), which has a vapor pressure of 97 mm Hg at 20° C. In this regard, for example, the high vapor pressure organic solvent may comprises a vapor pressure, for example, of at least about 20 mm HG at 23° C., at least about 50 mm Hg at 23° C., at least about 100 mm Hg at 23° C., at least about 150 mm HG at 23° C., at least about 200 mm Hg at 23° C., or at least about 300 mm Hg at 23° C.

Upon drying (or allowing to dry) of the formed nanofibers, pores with a diameter ranging, for example, of a few nanometers will be formed due to the evaporation of the high vapor pressure organic solvent, such as 1,2-dichloroethane (DCE). For example, the nanofibers shown in FIG. 4 were formed utilizing PLGA as a biodegradable polymer and DCE as a high vapor pressure organic solvent in an electrospinning process. Depending on the concentration and spinning conditions, the average pore sizes (e.g., diameter and/or depth) on the nanofiber outer surface can be tailored as desired. In accordance with certain embodiments of the present invention, an acid scavenger may be formulated into the biodegradable polymer matrix forming the nanofibers. The acid scavenger, for example, may facilitate or eliminate the accumulation of acidic by-products from the biopolymers (e.g., biodegradable polymers). The acid scavengers utilized, according to certain embodiments of the present invention, are not particularly limited, but may include Ca₃(PO₄)₂ or the like.

Once the porous nanofibers have been formed, target therapeutic agent(s) can be infused onto the nanofibers, for example, through a soaking or pressure induced diffusion process in which a therapeutic compositing comprising one or more target therapeutics are infused within the plurality of pores of the nanofibers to provide drug-eluting nanofibers. In accordance with certain embodiments of the present invention, for example, the plurality of pores comprise a tuned average pore size (e.g., diameter and/or depth) and/or pore size distribution as determined for a desired rate of release for the one or more therapeutic agent contained within the pores. In this regard, the drug-eluting nanofibers may provide a desired controlled, delayed, and/or sustained release of the one or more therapeutic agents one positioned in a mammalian body.

Although the average pore size (e.g., diameter and/or depth) of the plurality of pores can be varied based on the desired therapeutic-release profile, the plurality of pores may comprise an average diameter, an average depth, or both from at least about any of the following: 0.1, 0.2, 0.3, 0.5, 1, 2, 3, 5, 25, 50, 75, and 100 nm and/or at most about 1000, 800, 700, 600, 500, 400, 300, 200 and 100 nm (e.g., about 0.1-1000 nm, about 5-75 nm, about 5-1000 nm, etc.).

Porous drug-eluting nanofibers, according to certain embodiments of the present invention, may comprise an outer diameter of less than about 10,000 nm. In certain embodiments, the outer diameter may comprise from at least about any of the following: 1, 5, 25, 100, and 1000 nm and/or at most about 10000, 5000, 2500, and 1000 nm (e.g., about 1-10000 nm, about 1-1000 nm, etc.).

In another aspect, the present invention provides a fabric (e.g., a nonwoven fabric) comprising a plurality of drug-eluting nanofibers as discussed above. In certain embodiments, the plurality of drug-eluting nanofibers comprise one or more therapeutic agents therein (e.g., within a core portion of sheath-core nanofibers, within an interior cavity of hollow nanofibers, within pores of porous nanofibers). In this regard, the fabric may comprise a drug-eluting fabric which may be formed into a variety of configurations (e.g., sleeve, tube, etc.) and attached and/or positioned onto or over the top of a medical device (e.g., a stent or graft), or used independently as a tissue engineering scaffold. Drug-eluting fabrics according to certain embodiments may comprise a mixture of drug-eluting nanofibers and non-drug-eluting fibers. In accordance with certain embodiments, the drug-eluting fabrics may be consolidated by light thermal bonding (e.g., light calendaring, which may also provide one or more seals if desired), ultrasonic bonding, mechanical bonding (e.g., light hydroentanglement), or adhesive bonding. In this regard, drug-eluting fabrics according to certain embodiments of the present invention may comprise one or more layers of fabric. In some embodiments, for instance, a drug-eluting fabric comprising a single layer of material may be joined with another layer of fabric (e.g., spunbond, meltblown, etc,), which provides support and strength to the layer of drug-eluting nanofibers, to form a drug-eluting composite. In this regard, the drug-eluting composite may comprise a first side comprising a drug-eluting layer of nanofibers in accordance with certain embodiments disclosed herein, and a second side comprising a support or strength fabric (e.g., spunbond, meltblown, etc,). The strength or support fabric may be selected to provide the necessary robustness for handling purposes, for example, when attached to a medical device that is or will be maneuvered through various body cavities and/or subjected to various medical procedures. In this regard, the strength or support fabric of the composite may be positioned adjacent or closest to an outer surface of a medical device, while the drug-eluting layer of the fabric will remain on the body-facing side of the composite. Such embodiments for instance, may mitigate the tearing and/or loss of a portion (or all) of the drug-eluting fabric from a medical device during implantation, while maintaining the drug-eluting properties desired.

In certain embodiments of the present invention, such as when used independently as a tissue engineering scaffold, the nanofiber fabric may contain nanofibers that elute different drugs (e.g., one or more therapeutic agents) depending on the location within the fabric (e.g., different layers or different regions within a layer may elute differing therapeutic agent(s)). Since traditionally implanted scaffolds are typically limited in size and exposed surface area, scaffolds comprising a plurality of drug-eluting nanofibers, according to certain embodiments of the present invention, provide a significantly greater exposed surface area. In accordance with certain embodiments of the present invention, the nanofiber fabric may comprise (or be used) as a tissue engineering scaffold in which the drug elution profile of each individual fiber (or groups of fibers) can be separately controlled. The vastly increased drug-eluting surface area, according to certain embodiments of the present invention, increases the amount of attractant biomolecules that can be released. More importantly, because different fibers within the same structure can be made to release different biomolecules, such a device will allow different parts of the scaffolds to be populated with different celltypes, potentially accelerating the repopulation of the scaffold in an appropriate anatomic configuration.

In accordance with certain embodiments, the present invention provides a tissue engineering scaffold comprising a drug-eluting fabric as disclosed herein. In certain embodiments, for example, the tissue engineering scaffold comprises at least a first drug-eluting fiber (or group of fibers) configured for releasing a first therapeutic agent (or group of therapeutic agents) and a second drug-eluting fiber (or group of fibers) configured for releasing a second therapeutic agent (or group of therapeutic agents). In certain embodiments, for instance, the first drug-eluting fiber and the second drug-eluting fiber are located at different regions (e.g., discrete and separate portions) of the tissue engineering scaffold. Tissue engineering scaffolds according to certain embodiments of the present invention, for example, may comprise a plurality of layers in which the first drug-eluting fiber is located within a first layer of the tissue engineering scaffold and the second drug-eluting fiber is located within a second layer of the tissue engineering scaffold.

### II. MEDICAL DEVICES

In another aspect, the present invention provides devices comprising a medical device (e.g., a stent, graft, etc.) and a plurality of drug-eluting nanofibers directly or indirectly located over and/or directly or indirectly joined to at least a portion of an outer surface of the medical device. As discussed previously, the plurality of drug-eluting nanofibers comprise one or more therapeutic agents that may be released in a desired therapeutic-releasing profile.

As noted above, the plurality of drug-eluting nanofibers may comprise one or more biodegradable polymers. In this regard, certain embodiments of the present invention comprise a device which provides a controlled, sustained, and/or delayed release of one or more therapeutic agents when positioned within a mammal. In accordance with certain embodiments of the present invention, the plurality of drug-eluting nanofibers may be directly deposited onto an outer surface of a medical device. Additionally or alternatively, the plurality of drug-eluting fibers can comprise a drug-eluting fabric or composite as discussed above. For example, a plurality of drug-eluting fibers can be formed into a fabric (e.g., a drug-eluting fabric), which can be configured into a variety of shapes or configurations (e.g., sleeve, tube, etc.). In this regard, the fabric or composite comprising a plurality of drug-eluting fibers can be directly or indirectly attached to a medical device during or prior to implantation of the medical device in a mammal.

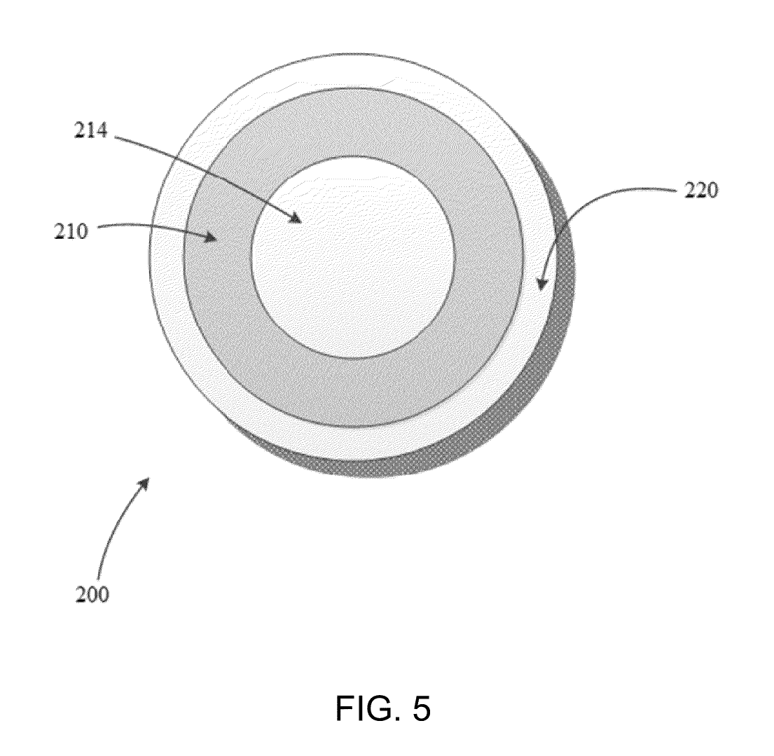

In certain embodiments, the medical device comprises a stent or graft. FIG. 5, for example, illustrates a device 200 according to certain embodiments of the present invention comprising a stent structure 210 defining an internal lumen 214. As illustrated in FIG. 5, the device 200 includes a plurality of drug-eluting fibers 220 in the form of a nonwoven fabric overlying the stent structure 210. The plurality of drug-eluting fibers 220 may comprise a variety of structures configured for releasing one or more therapeutic agents when disposed within a mammalian body. In certain embodiments, for example, the plurality of drug-eluting nanofibers comprise a sheath-and-core configuration, as discussed in detail above, comprising a core and a sheath surrounding the core, in which the core comprises one or more therapeutic agents. As noted above, the core and the sheath may each comprise a biodegradable polymer or polymers, in which the core may comprise the same or different biodegradable polymer or polymers of the sheath.

In certain embodiments of the present invention, the plurality of drug-eluting fibers, which may be directly or indirectly located over the medical device, may comprise a hollow-fiber configuration comprising an outer wall defining at least one interior cavity as discussed in detail above. As noted above, one or more therapeutic agents may be located at least in the interior cavity. For instance, the hollow portion of the nanofiber may a therapeutic composition comprising the one or more therapeutic agents. For instance, the therapeutic composition may comprise a solution, suspension, gel, solid-preparation or any combinations thereof. As noted above, the plurality of drug-eluting nanofibers may comprise one or more seals (e.g., a mechanical crimp, thermally formed seal, etc.) configured to temporarily contain the therapeutic composition within the interior cavity.

Devices according to certain embodiments may comprise drug-eluting nanofibers comprising a porous-outer surface comprising a plurality of pores as discussed in detail above. For instance, the plurality of pores comprise one or more therapeutic agents disposed therein. The plurality of pores may, for instance, comprise a general pit-like or bowl-like structure being suitable for housing or containing a therapeutic composition (e.g., one or more therapeutic agents) therein.

Devices in accordance with certain embodiments of the present invention may comprise a plurality of drug-eluting nanofibers directly or indirectly located over and/or directly or indirectly joined to at least a portion of an outer surface of a medical device. Although a stent or graft has been utilized primarily throughout the present specification, such use is merely exemplary and non-limiting. As noted above, for instance, the term “medical device” may comprise a wide range of medical devices, such as any medical device capable of being inserted into a mammalian (e.g., human) body and used in conjunction with a plurality of drug-eluting fibers (e.g., nanofibers). Medical devices, for example, may comprise stents, stent sleeves, pacemakers, vascular grafts, implantable cardioverter-defibrillators, pacemaker electrodes, implantable cardioverter-defibrillator leads, biventricular implantable cardioverter-defibrillator leads, artificial hearts, artificial valves, ventricular assist devices, balloon pumps, catheters, central venous lines, implants, or sensors.

### III. EXAMPLES

The present disclosure is further illustrated by the following examples, which in no way should be construed as being limiting. That is, the specific features described in the following examples are merely illustrative and not limiting.

Structures according to certain embodiments of the present invention, the diffusion of water molecules through such structures can be estimated based on Fick's second Law:

$$\frac{\partial C}{\partial t} = D \frac{{\partial }^{2} C}{\partial {x}^{2}} \frac{C ( x , t ) - {C}_{0}}{Cs - {C}_{0}} = 1 - erf ( \frac{x}{2 \sqrt{Dt}} )$$

wherein,

D is determined from Stokes-Einstein relation:

$$D = \frac{kT}{6 π r η}$$

η: dynamic viscosity of solute (Pa·s)

k: Boltzmann constant (i.e., 1.38×10⁻²³ J/K)

T: temperature (K)

R: radius of solute molecules

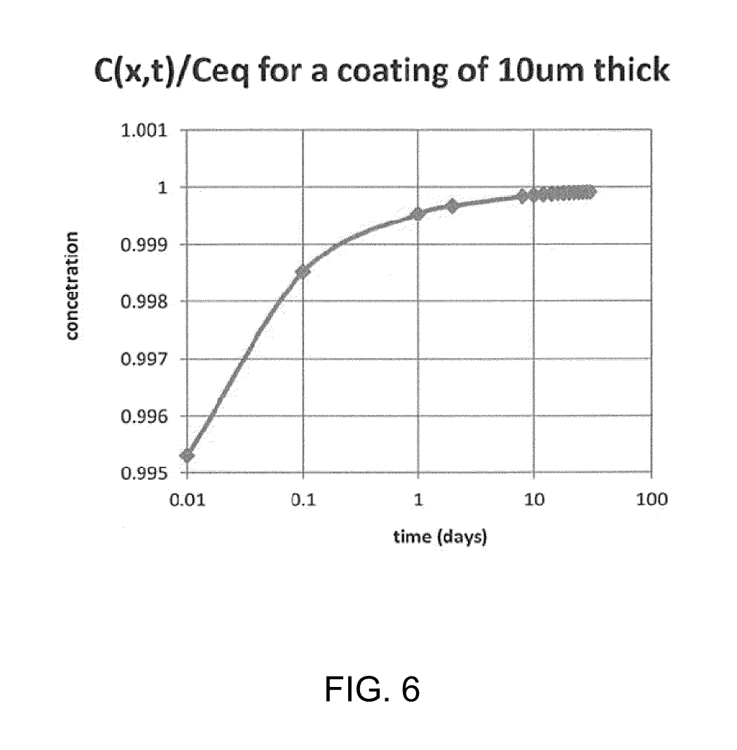

FIG. 6 illustrates the estimated diffusion kinetics of water diffusing through a 10 micron coating of biodegradable polymer, which illustrates that the diffusion through structures according to certain embodiments of the present invention can comprise several days.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A device, comprising:
(a) a medical device; and
(b) a plurality of drug-eluting nanofibers directly or indirectly located over an outer surface of the medical device; wherein the plurality of drug-eluting nanofibers comprise one or more therapeutic agents.

2. The device according to claim 1, wherein the plurality of drug-eluting nanofibers comprise a sheath-and-core configuration comprising a core and a sheath surrounding the core; wherein the core comprises the one or more therapeutic agents.

3. The device of claim 2, wherein the core and the sheath each comprise a biodegradable polymer; said core may comprise the same or different biodegradable polymer of the sheath.

4. The device of claim 1, wherein the plurality of drug-eluting nanofibers comprise a hollow-fiber configuration comprising an outer wall defining an interior cavity.

5. The device of claim 4, wherein the one or more therapeutic agents is located at least in the interior cavity.

6. The device of claim 4, wherein the interior cavity comprises a therapeutic composition comprising the one or more therapeutic agents.

7. The device of claim 6, wherein the therapeutic composition comprises a solution, suspension, gel, solid-preparation or any combinations thereof.

8. The device of claim 7, wherein the plurality of drug-eluting nanofibers comprise one or more seals configured to temporarily contain the therapeutic composition within the interior cavity.

9. The device of claim 8, wherein the one or more seals comprise a mechanical crimp, thermally formed seal, or combination thereof.

10. The device of claim 1, wherein the plurality of drug-eluting nanofibers comprise a porous-outer surface comprising a plurality of pores.

11. The device of claim 10, wherein the plurality of pores comprise the one or more therapeutic agents disposed therein.

12. The device of claim 11, wherein the plurality of pores comprise an average diameter, an average depth, or both from about 5 nm to about 1000 nm.

13. The device of claim 11, wherein the plurality of pores comprise pit-like structure.

14. The device of claim 1, wherein the plurality of drug-eluting nanofibers comprise a nonwoven fabric positioned directly or indirectly over the outer surface of the medical device.

15. The device of claim 1, wherein the nonwoven fabric comprises a sleeve.

16. A fabric, comprising a plurality of drug-eluting nanofibers, wherein the plurality of drug-eluting nanofibers comprise one or more therapeutic agents.

17. The fabric of claim 16, wherein the plurality of drug-eluting nanofibers comprise a sheath-and-core configuration, a hollow-fiber configuration, a porous-outer surface comprising a plurality of pores, or any combination thereof.

18. A tissue engineering scaffold, comprising a fabric, wherein the fabric comprises a plurality of drug-eluting nanofibers, and wherein the plurality of drug-eluting nanofibers comprise one or more therapeutic agents.

19. The tissue engineering scaffold of claim 18, wherein the tissue engineering scaffold comprises at least a first drug-eluting fiber configured for releasing a first therapeutic agent and a second drug-eluting fiber configured for releasing a second therapeutic agent.

20. The tissue engineering scaffold of claim 19, wherein the first drug-eluting fiber is located within a first layer of the tissue engineering scaffold and the second drug-eluting fiber is located within a second layer of the tissue engineering scaffold.
